(19) United States
(12) Patent Application Publication
(10) Pub. No.: US 2026/0102274 A1
(43) Pub. Date: Apr. 16, 2026

(54) Penile Shaping Device

(71) Applicant: Sergio Castaneda, Las Vegas, NV (US)

(72) Inventor: Sergio Castaneda, Las Vegas, NV (US)

(21) Appl. No.: 19/328,148

(22) Filed: Sep. 14, 2025

Related U.S. Application Data
(60) Provisional application No. 63/705,511, filed on Oct. 10, 2024.

(51) Int. Cl. A61F 5/41

(52) U.S. Cl. CPC A61F 5/41; A61F 2005/411; A61F 2005/412

(57) ABSTRACT

An improved penile shaping device having an attachable proximal end with an opening capable of interfacing with a human body, a cylindrical frame unit, the cylindrical frame unit including one or more cylindrical section units, the cylindrical section units having angled ends; and an attachable distal end. The attachable proximal end is attached to the proximal end of the cylindrical frame unit and the attachable distal end is attached to the distal end of the cylindrical frame unit. The cylindrical frame unit having one or more segments creates one or more bends in the cylindrical frame unit when the one or more segments is rotated. A shaping force is applied to an inserted penis by creating an angled cylindrical frame unit by rotating one or more cylindrical section units and applying a partial vacuum to the device.

### CROSS-REFERENCE TO RELATED APPLICATIONS

The present nonprovisional patent application claims priority benefit of the U.S. provisional application for patent Ser. No. 63/705,511 titled “Improved Penile Shaping Device” filed on Oct. 9, 2024 under 35 U.S.C. 119(e). The contents of this related application are incorporated herein by reference for all purposes to the extent that such subject matter is not inconsistent herewith or limiting hereof.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### BACKGROUND OF THE INVENTION

### 1. Field of the Invention

One or more embodiments of the invention generally relate to penile shaping devices. More particularly, embodiments of the invention relate to an improved penile shaping, straightening, and enhancement device capable of being implemented for treatment of medical conditions ranging from erectile dysfunction to penile injury to Peyronie's disease.

### 2. Description of the Related Art

Male erectile dysfunction is a common medical problem. It has many roots both physiological and psychological, but its effects may be devastating for both the individual and the individual's partner. Erectile dysfunction and/or penile shortening may be the result of common aging to surgical procedures such as radical prostatectomy and transurethral resection of the prostate.

Peyronie's disease (PD) is a connective tissue disorder involving the tunica albuginea layer of the penis. It is a condition that develops over time, and causes an abnormal curvature of the penis when erect. The fibrotic process that occurs may result in the formation of plaque, one of the classic findings on physical examination of the patient. Men afflicted by this disorder may present with pain, deformity (such as penile curvature or penile shortening) and/or erectile dysfunction (ED).

Several methods of treatment for penile injuries and Peyronie's disease are now in use ranging from drug therapy to the use of medical devices. In addition to erectile dysfunction treatment, many males wish to enhance their penis size and sexual performance, to which analogous treatments are available.

Among the more extreme methods of treatment for penile injury and Peyronie's disease is the use of relatively uncomfortable medical devices which can cause severe discomfort. Surgical procedures and injection of collagenase enzymes are also available. Though proven effective, these methods of treatment come with serious risks which deter numerous men from seeking such treatment; and while surgical therapy has proven itself to be a definitive treatment for penile curvature associated with Peyronie's disease, less invasive options are currently a desired alternative.

Penile traction devices are a class of medical device for treating erectile dysfunction and for increasing penile length. These devices usually consist of a plastic support ring, a silicone band, and two dynamic rods. Such therapy works by holding the penis in a cradle and subjecting it to gentle and progressive shaping forces that can be achieved by the addition of small metal extensions to the dynamic rods and cradle frame every few weeks. As the penile tissues are slowly yet surely stretched, which causes microscopic tears which, in theory, the body heals by producing cells to repair the gap. With each repair phase, the penile tissues expand and form a straighter erect penis.

Penile traction devices have limitations though. Most notably, penile traction devices can be highly uncomfortable and painful to use. Presently, there exists a need for an improved penile shaping device which is capable of providing the results of existing traction devices, but is more comfortable to use and is capable of producing better results.

### SUMMARY

The present invention introduces a new technology, which fulfils the need for a more versatile penile shaping device which provides greater comfort and reduced anxiety for a user. At its essence, the improved penile shaping device comprises a plurality of outer and inner elements which create a vacuum tube and a plurality of shaping elements when assembled. The improved penile shaping device may also include various other therapeutic implementations such as traction and electrotherapeutic devices. The improved penile shaping device and method is versatile and readily implementable to treat a wide variety of penile injury problems and may serve to enhance penis length and girth.

The invention is primarily directed to an improved penile shaping device comprising an attachable proximal end having an opening capable of interfacing with a human body, a cylindrical frame unit, the cylindrical frame unit comprising one or more cylindrical section units, the cylindrical section units having angled ends; and an attachable distal end. The attachable proximal end is attached to the proximal end of the cylindrical frame unit and the attachable distal end is attached to the distal end of the cylindrical frame unit. The cylindrical frame unit having one or more segments creates one or more bends in the cylindrical frame unit when the one or more segments is rotated.

In embodiments of the invention, the proximal end capable of interfacing with a human body is angled. The angle of the said proximal end may vary depending on patient needs. An angled proximal end may serve to shape a penis when inserted by virtue of the angle itself, or the penis can be further shaped by a user applying manual or gravimetric force to the assembled penile shaping device.

In embodiments of the invention, the distal end has one or more attachment points for one or more accessories to attach to the said distal end. In some embodiments, the one or more accessories is a weighted attachment. In other embodiments, the distal end itself may be a weighted attachment.

In embodiments of the invention, a vacuum pump assembly may be detachably affixed to the either the proximal end, the cylindrical frame unit, or distal end in more than one way, so that when attached and connected with a length of tubing, the pump assembly may be easily worked with a weighted distal end attached to the cylindrical frame unit. The present invention is intended to be used with numerous pumps available on the market. This feature provides two operational attributes in one device, both a vacuum device and a traction device.

The partial vacuum created in the device draws blood into the penile tissues, inducing an erection-like state. For treatment of Peyronie's Disease, the object is to mechanically stretch and shape a penis, potentially reducing curvature, improving penile length, and preserving and/or enhancing erectile function.

In embodiments of the present invention, the improved penile shaping device can be used with various shaping and vacuum therapy methods. The invention is also capable of being used with a variety of vacuum devices, thus providing multiple operational attributes in one device, both a vacuum device and a shaping device.

Use of the improved penile shaping device involves the user assembling the vacuum tube, preparing and inserting a penis into an assembled vacuum tube, securing the penis in the vacuum tube, applying a partial vacuum to engorge a penis, and moving the shaping elements in place so as to straighten a penis. Additional steps may include, but are not limited to, applying a mild electrical current for a therapeutic amount of time.

### BRIEF DESCRIPTION OF THE DRAWINGS

The invention directed by way of example, and not by way of limitation, in the figures of the accompanying drawings and in which like reference numerals refer to similar elements and in which:

FIG. 1 is an exploded side view of the exemplary improved penile shaping device in accordance with an embodiment of the invention;

FIG. 2 is side view of an attachable proximal end of the improved penile shaping device in accordance with an embodiment of the invention;

FIG. 3 is a front view of an attachable proximal end of the improved penile shaping device in accordance with an embodiment of the invention;

FIG. 4 is an exploded perspective view of the exemplary penile shaping device in accordance with an embodiment of the invention;

FIG. 5 is a cross sectional view of the exemplary penile shaping device in accordance with an embodiment of the invention;

FIG. 6 is a cross sectional view of the exemplary penile shaping device in accordance with an embodiment of the invention;

FIG. 7 is a perspective sectional view of the exemplary penile shaping device in accordance with an embodiment of the invention;

FIG. 8 is a perspective view of the exemplary penile shaping device in accordance with an embodiment of the invention; and

FIG. 9 is a is a front view of the exemplary penile shaping device in accordance with an embodiment of the invention.

Unless otherwise indicated illustrations in the figures are not necessarily drawn to scale.

### DETAILED DESCRIPTION OF THE PREFERRED EMBODIMENTS

Terminology used herein is used for the purpose of describing particular embodiments only, and is not intended to limit the scope of the present invention. It must be understood that as used herein and in the appended claims, the singular forms “a,” “an,” and “the” include the plural reference unless the context clearly dictates otherwise. For example, a reference to “an element” is a reference to one or more elements and includes all equivalents known to those skilled in the art. All conjunctions used are to be understood in the most inclusive sense possible. Thus, the word “or” should be understood as having the definition of a logical “or” rather than that of a logical “exclusive or”unless the context clearly necessitates otherwise. Language that may be construed to express approximation should be so understood unless the context clearly dictates otherwise.

Unless defined otherwise, all technical and scientific terms used herein have the same meanings as commonly understood by a person of ordinary skill in the art to which this invention belongs. Preferred methods, techniques, devices, and materials are described. But any methods, techniques, devices, or materials similar or equivalent to those described herein may be used in the practice or testing of the present invention. Structures described herein should also be understood to refer to functional equivalents of such structures.

A “cylindrical segment,” sometimes also called a “truncated cylinder,” is the solid cut from a circular cylinder by two (or more) planes. If there are two cutting planes, one perpendicular to the axis of the cylinder and the other tilted with respect to it, the resulting solid is known as a “cylindrical wedge.” If the plane is titled with respect to a circular cross section but does not cut the bottom base, the resulting cylindrical segment has one circular cap and one elliptical cap. It is a circle (if the plane is at a right angle to the axis), an ellipse, or, if the plane is parallel to the axis, a single line (if the plane is tangent to the cylinder), pair of parallel lines bounding an infinite rectangle (if the plane cuts the cylinder), or no intersection at all (if the plane misses the cylinder entirely.

A “gasket” is a mechanical seal that is placed between two surfaces to prevent leakage of fluids or gases. It essentially fills the gap between the surfaces, creating a pressure-tight seal.

References to “one embodiment,” “an embodiment,” “various embodiments,” etc., may indicate that the embodiment(s) of the invention so described may include particular features, structures, or characteristics. However, not every embodiment necessarily includes the particular features, structures, or characteristics. Further, repeated use of the phrase “in one embodiment,” or “in an exemplary embodiment,” do not necessarily refer to the same embodiment although they may. A description of an embodiment with several components in communication with each other does not imply that all such components are required. On the contrary, a variety of optional components are described to illustrate the wide variety of possible embodiments of the present invention.

As is well known to those skilled in the art, many careful considerations and compromises typically must be made when designing for the optimal manufacture of a commercial implementation of such an improved penile shaping device. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The exemplary improved penile shaping device will now be described in detail with reference to embodiments thereof as illustrated in the accompanying drawings.

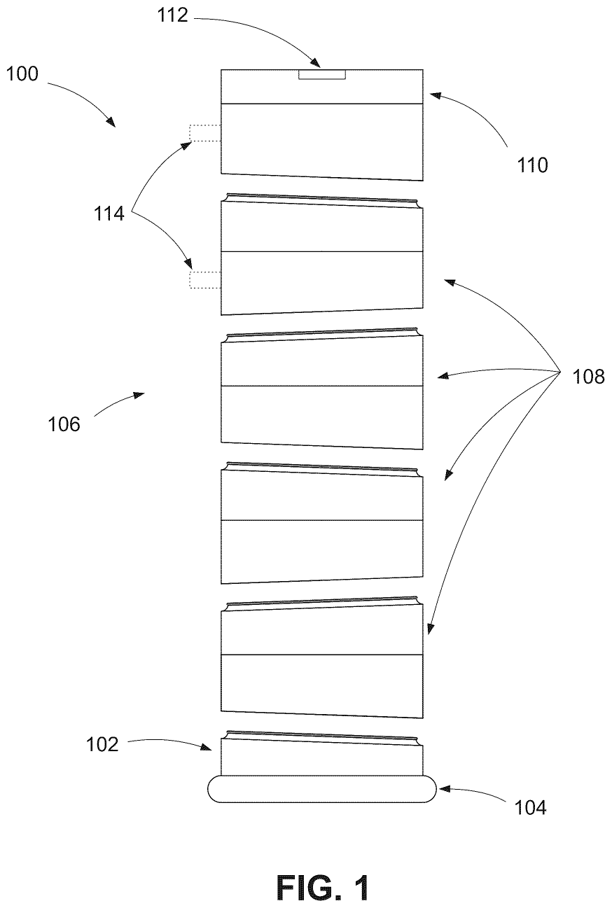

FIG. 1 is an exploded side view of the exemplary improved penile shaping device in accordance with an embodiment of the invention. The invention includes an attachable proximal end 102 which includes an opening for a human penis. The attachable proximal end is capable of interfacing with a human body which includes the immediate area surrounding a penis, also known as the pubic area. The attachable proximal end has an opening 104 which can serve as a sealing flange when the attachable proximal end is placed against the pubic area.

The invention further includes a cylindrical frame unit 106 comprising one or more cylindrical section units 108. The cylindrical frame units have angled ends which, when the cylindrical frame units are rotated, allow for the cylindrical frame to assume different angles. Such angles allow for shaping of a penis when inserted into the penile shaping device.

The cylindrical section units 108 which serve as a component which can be rotated in numerous ways so as to provide greater or lesser angles so as to create a shaping force for an inserted penis when the cylindrical section units 108 are rotated. In some embodiments of the invention, further includes outer body units which may serve as an additional rigid outer component which, when assembled with the inner body units 108 form a vacuum cylinder which includes elements for shaping a penis when inserted into the assembled vacuum cylinder.

The invention further includes an attachable distal end 110. The attachable distal end includes at least one attachment point 112 for one or more accessories to attach to the said distal end.

The attachable proximal end 102 can include one or more vacuum line attachments. The cylindrical section units 108 can include one or more vacuum line attachments 114. The attachable distal end 112 can include one or more vacuum line attachments 114. Persons skilled in the art will appreciate that a vacuum line attachment is typically a valve mechanism known and appreciated in the art. The valve mechanism pairs with a vacuum pump wherein the vacuum pump creates a partial vacuum in the cylinder. A vacuum line attachment can be located on other parts of the invention such as, but not limited to, attachments for the attachable distal end.

In embodiments of the invention, the distal end 112 has one or more attachment points for one or more accessories to attach to the distal end. In some embodiments, the one or more accessories is a weighted attachment. In some embodiments, the attachment can be a device configured for extracorporeal shock therapy. In other embodiments, the distal end itself may be a weighted attachment.

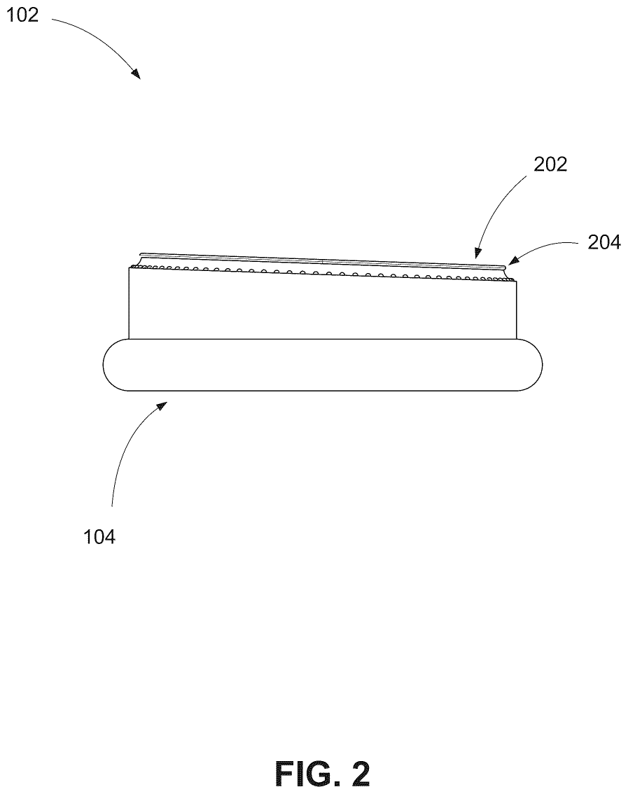

FIG. 2 is side view of an attachable proximal end of the improved penile shaping device in accordance with an embodiment of the invention. The attachable proximal end is configured to interface with a human body by way of the immediate area surrounding a penis, also known as the pubic area. In the preferred embodiment, the attachable angled proximal end is cylindrically shaped, and made of a plastic housing which may be made of a clear, medical-grade polycarbonate resin plastic. Persons skilled in the art will readily appreciate that alternative embodiments may be made from a number of materials such as, but not limited to, clear acrylic plastics, ABS plastic, polycarbonates, polyvinyl chloride (PVC), or various other plastic equivalents.

The attachable proximal end 102 has an opening 104 which can serve as a sealing flange when the attachable proximal end is placed against the pubic area. The opening 104 may include a sealing constriction ring which is attachably mounted on the attachable proximal end in the vicinity of the fitted opening 104. The attachable proximal end 102 is a generally ring-shaped body having a circumferentially extending flange for releasable sealing engagement with the pubic area. In the preferred embodiment, the opening 104 will be round and circumferentially shaped. However, persons skilled in the art will appreciate that other shapes and configurations for the opening can be utilized.

In this view, the attachable proximal end 102 is depicted as having a one hundred and eighty-degree angle. However, in other embodiments of the present invention, the angle may vary. In one embodiment of the invention, a kit is provided with a forty-five degree angled proximal end attachment, a ninety-degree proximal end attachment, and a one-hundred and eighty degree attachment. In one exemplary embodiment, a kit is provided with ten-, twenty-, thirty-, forty-five, sixty-and ninety-degree attachments. The purpose of the angled attachments is to allow the user differing angles of distraction therapy coupled with preventing painful stress on the testicles and other sensitive areas.

The attachable proximal end 102 includes a joint mechanism 202 which attaches the attachable proximal end with the cylindrical frame unit comprising one or more cylindrical section units. In one exemplary embodiment of the invention, the joint mechanism 202 is an annular snap fit joint including a male component and a female component. In this figure, a male component is shown. The male component mates with a female component in which the male component has a bump or ridge 204 around the circumference of the male component which fits in a groove in a female component. In some embodiments of the invention, the male component is made from a more rigid material than the female component. This allows the female component to flex and deform as the male part is inserted. By way of example, the joint mechanism 202 allows for the cylindrical sections which comprise the penile shaping device 100 to rotate. In other embodiments, a thread mechanism can be used. However, persons skilled in the art will appreciate that other such joint mechanisms 202 may be utilized.

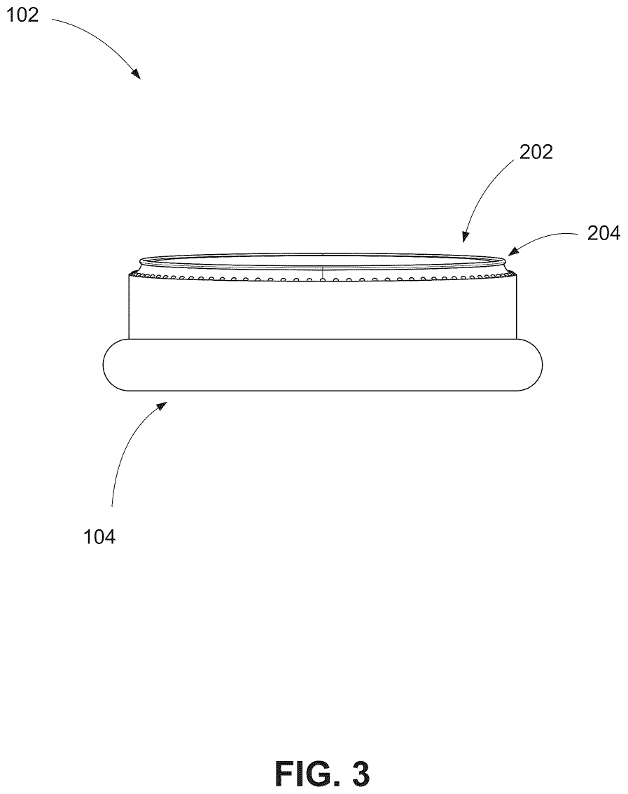

FIG. 3 is a front view of an attachable proximal end of the improved penile shaping device in accordance with an embodiment of the invention. The attachable proximal end is capable of interfacing with a human body which includes the immediate area surrounding a penis, also known as the pubic area. The attachable proximal end has an opening 104 which can serve as a sealing flange when the attachable proximal end is placed against the pubic area.

The attachable proximal end 102 includes a joint mechanism 202 which attaches the attachable proximal end with the cylindrical frame unit comprising one or more cylindrical section units. In one exemplary embodiment of the invention, the joint mechanism 202 is an annular snap fit joint including a male component and a female component. In this figure, a male component is shown. The male component mates with a female component in which the male component has a bump or ridge around the circumference of the male component which fits in a groove in a female component. By way of example, the joint mechanism 202 allows for the sections which comprise the penile shaping device 100 to rotate. In other embodiments, a thread mechanism can be used. However, persons skilled in the art will appreciate that other such joint mechanisms 202 may be utilized.

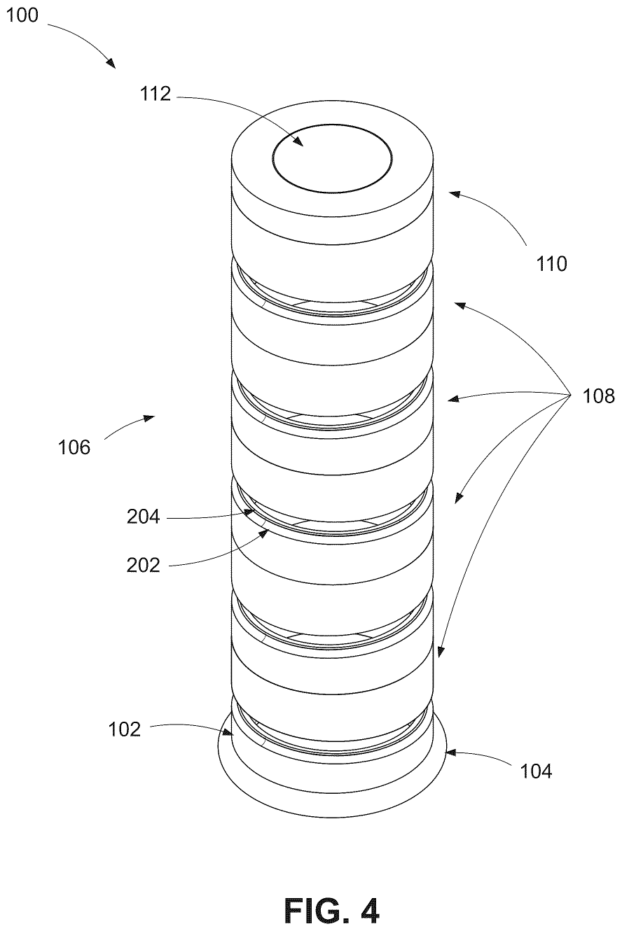

FIG. 4 is an exploded perspective view of the exemplary penile shaping device in accordance with an embodiment of the invention. The invention includes an attachable proximal end 102 which includes an opening for a human penis. The attachable proximal end is capable of interfacing with a human body which includes the immediate area surrounding a penis, also known as the pubic area. The attachable proximal end has an opening 104 which can serve as a sealing flange when the attachable proximal end is placed against the pubic area.

The invention further includes a cylindrical frame unit 106 comprising one or more cylindrical section units 108. In this figure, four cylindrical section units 108 are used. However, other embodiments of the invention can include a greater or lesser number of cylindrical section units. Each cylindrical section units 108 includes a joint mechanism 202 which allows the cylindrical section units to attach to one another. The cylindrical frame units have angled ends which, when the cylindrical frame units are rotated, allow for the cylindrical frame to assume different angles. Such angles allow for shaping of a penis when inserted into the penile shaping device. In this figure, a male component is shown. The male component mates with a female component in which the male component has a bump or ridge 204 around the circumference of the male component which fits in a groove in a female component. In some embodiments of the invention, the male component is made from a more rigid material than the female component. This allows the female component to flex and deform as the male part is inserted. By way of example, the joint mechanism 202 allows for the cylindrical sections which comprise the penile shaping device 100 to rotate. In other embodiments, a thread mechanism can be used. However, persons skilled in the art will appreciate that other such joint mechanisms 202 may be utilized.

A gasket means is used to create a seal between segments. In some embodiments of the invention, a gel sealant such as petroleum jelly or other oil is used. In other embodiments, an O-ring mechanism can be used. Persons skilled in the art will appreciate that numerous means and methods can be utilized to create a gasket between the attachable proximal end 102 and a cylindrical section unit 108. Likewise, numerous means and methods can be utilized to create a gasket between the cylindrical section units 108. Moreover, numerous means and methods can be utilized to create a gasket between a cylindrical section unit 108 and an attachable distal end. The optimal gasket means would be a material which allowed to the cylindrical section units 108 to freely rotate while allowing a user to create a partial vacuum in an assembled penile shaping device 100.

The cylindrical section units 108 which serve as a component which can be rotated in numerous ways so as to provide greater or lesser angles so as to create a shaping force for an inserted penis when the cylindrical section units 108 are rotated. In some embodiments of the invention, each cylindrical section unit 108 further includes outer body units which may serve as an additional rigid outer component which, when assembled with the cylindrical section units 108 form a vacuum cylinder which includes elements for shaping a penis when inserted into the assembled vacuum cylinder.

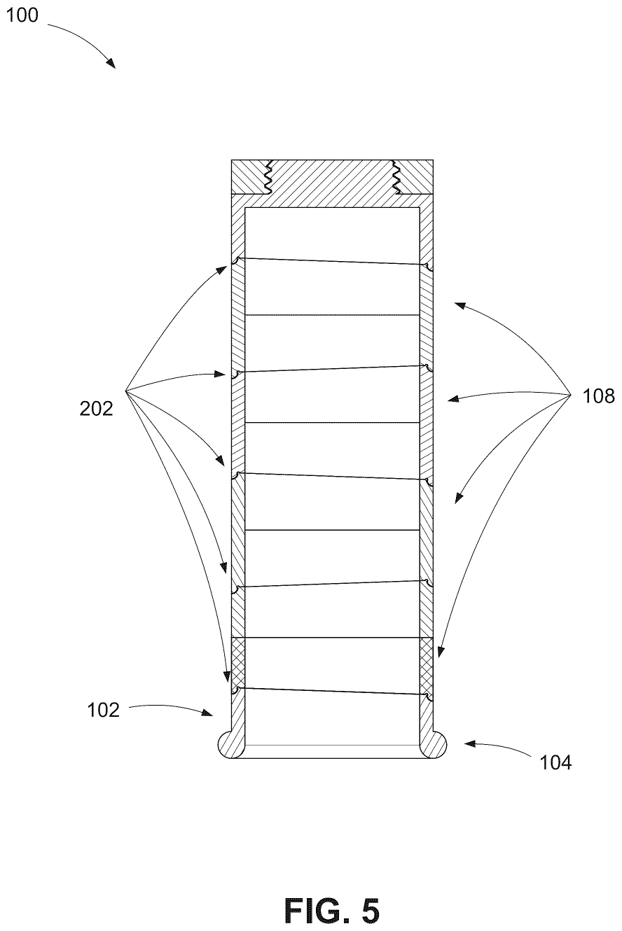

FIG. 5 is a cross-sectional view of the exemplary penile shaping device 100 in accordance with an embodiment of the invention. In such an embodiment, no cylindrical section units 108 have been rotated. The male component of each joint mechanism 202 mates with a female component in which the male component has a bump or ridge around the circumference of the male component which fits in a groove in a female component. As such, the penile shaping device 100 is straight.

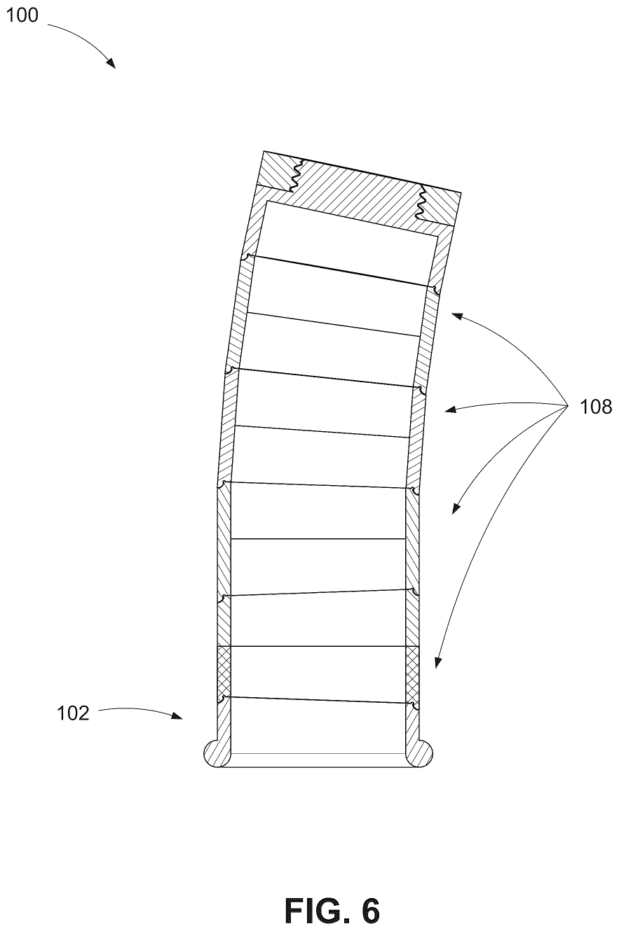

FIG. 6 is a cross-sectional view of the exemplary penile shaping device in accordance with an embodiment of the invention. In such an embodiment, each of the cylindrical section units 108 have been rotated a certain distance. Because the cylindrical section units are angled, the penile shaping device 100 no longer resembles a true cylinder. The penile shaping device is now bent in such a manner that when a penis is inserted, vacuum is applied and a penis becomes engorged and erect, a shaping force can be applied by way of a penis being “bent”in the same direction as the penile shaping device 100.

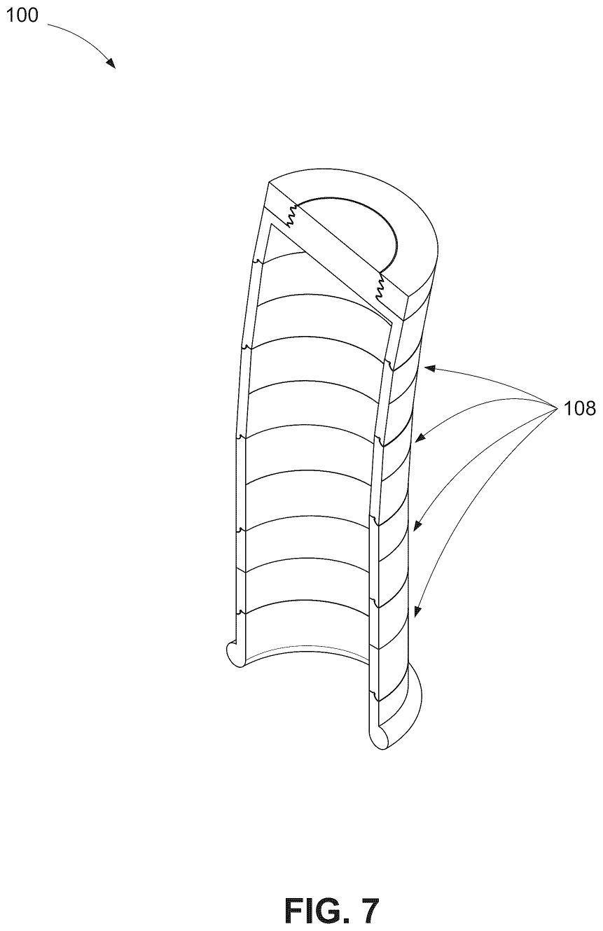

FIG. 7 is a perspective sectional view of the exemplary penile shaping device in accordance with an embodiment of the invention. In such an embodiment, each of the cylindrical section units 108 have been rotated a certain distance. Because the cylindrical section units are angled, the penile shaping device 100 no longer resembles a true cylinder. The penile shaping device is now bent in such a manner that when a penis is inserted, vacuum is applied and a penis becomes engorged and erect, a shaping force can be applied by way of a penis being “bent”in the same direction as the penile shaping device 100.

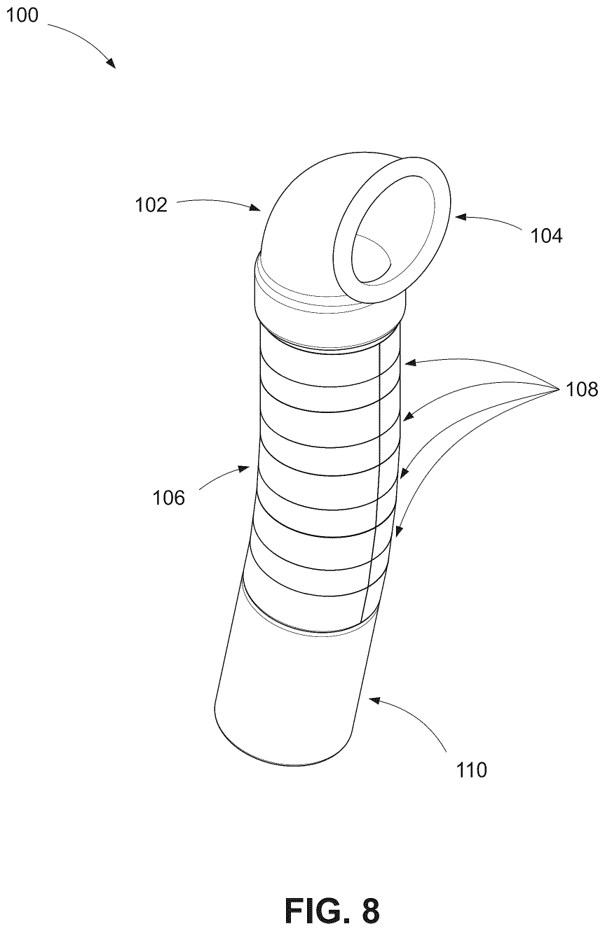

FIG. 8 is a perspective view of the exemplary penile shaping device in accordance with an embodiment of the invention. The invention includes a cylindrical frame unit 106 comprising one or more cylindrical section units 108. The cylindrical frame units have angled ends which, when the cylindrical frame units are rotated, allow for the cylindrical frame to assume different angles. Such angles allow for shaping of a penis when inserted into the penile shaping device.

The cylindrical section units 108 which serve as a component which can be rotated in numerous ways so as to provide greater or lesser angles so as to create a shaping force for an inserted penis when the cylindrical section units 108 are rotated. In some embodiments of the invention, further includes outer body units which may serve as an additional rigid outer component which, when assembled with the inner body units 108 form a vacuum cylinder which includes elements for shaping a penis when inserted into the assembled vacuum cylinder.

The attachable proximal end 102 has an opening 104 which can serve as a sealing flange when the attachable proximal end is placed against the pubic area. The opening 104 may include a sealing constriction ring which is attachably mounted on the attachable proximal end in the vicinity of the fitted opening 104. The attachable proximal end 102 is a generally ring-shaped body having a circumferentially extending flange for releasable sealing engagement with the pubic area. In the preferred embodiment, the opening 104 will be round.

In this view, the attachable proximal end 102 is depicted as having a ninety-degree angle. However, in other embodiments of the present invention, the angle may vary. In one embodiment of the invention, a kit is provided with a forty-five degree angled proximal end attachment, a ninety-degree proximal end attachment, and a one-hundred and eighty degree attachment. In one exemplary embodiment, a kit is provided with ten-, twenty-, thirty-, forty-five, sixty-and ninety-degree attachments. The purpose of the angled attachments is to allow the user differing angles of distraction therapy coupled with preventing painful stress on the testicles and other sensitive areas.

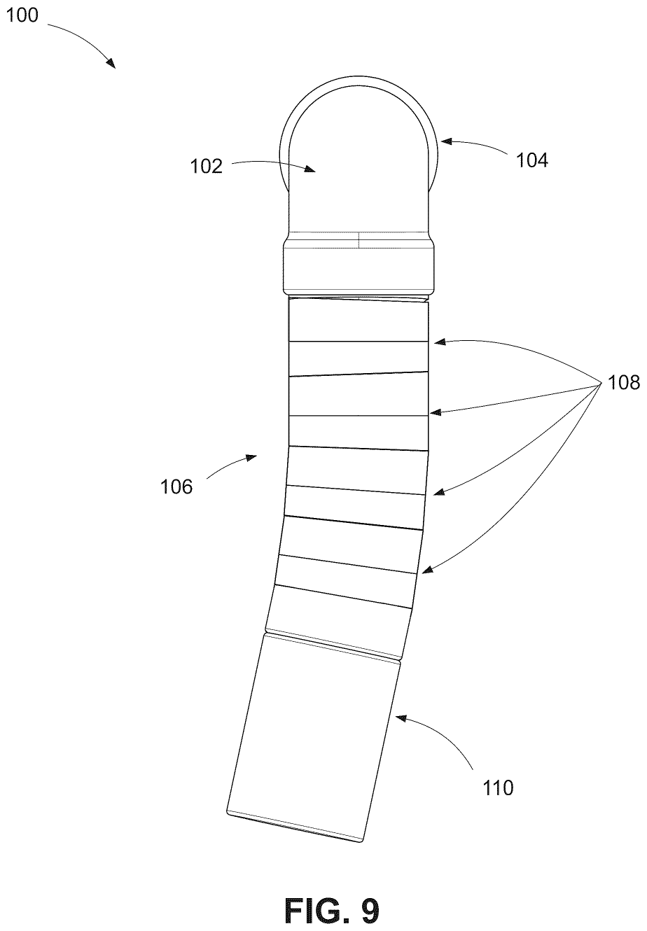

FIG. 9 is a is a front view of the exemplary penile shaping device in accordance with an embodiment of the invention. The invention includes a cylindrical frame unit 106 comprising one or more cylindrical section units 108. The cylindrical frame units have angled ends which, when the cylindrical frame units are rotated, allow for the cylindrical frame to assume different angles. Such angles allow for shaping of a penis when inserted into the penile shaping device.

The cylindrical section units 108 which serve as a component which can be rotated in numerous ways so as to provide greater or lesser angles so as to create a shaping force for an inserted penis when the cylindrical section units 108 are rotated. In some embodiments of the invention, further includes outer body units which may serve as an additional rigid outer component which, when assembled with the inner body units 108 form a vacuum cylinder which includes elements for shaping a penis when inserted into the assembled vacuum cylinder.

The attachable proximal end 102 has an opening 104 which can serve as a sealing flange when the attachable proximal end is placed against the pubic area. The opening 104 may include a sealing constriction ring which is attachably mounted on the attachable proximal end in the vicinity of the fitted opening 104. The attachable proximal end 102 is a generally ring-shaped body having a circumferentially extending flange for releasable sealing engagement with the pubic area. In the preferred embodiment, the opening 104 will be round.

Use of the improved penile shaping device is designed to be simple and minimally invasive. Use involves the user assembling the device 100, inserting a penis into the assembled device and creating a seal with the opening 104 of the invention. Various electrotherapeutic or extracorporeal shock therapeutic elements can be attached to a penis at this time. Once the penis is secured and the, the user is then able apply a vacuum to create an erect penis. With the penis erect, shaping force is applied to a secured penis by creating an angled cylindrical frame unit 106 by rotating one or more cylindrical section units 108. The cylindrical frame unit 106 can be shaped and moved so as to shape the erect penis while a vacuum is applied for a therapeutic amount of time. By attaching various attachable distal ends 110 to the penile shaping device 100, varying distractive force can be applied as well. Various electrotherapeutic treatment options can be applied at this time as well. Upon completion of the therapeutic amount of time, or when the user experiences discomfort, the device may be easily detached by loosening the cylindrical section units 108 and/or releasing vacuum and removing the penis from the device 100.

The exemplary improved penile traction device may be sold as a single unit or as a kit with interchangeable components. In some embodiments, a patient-specific fitted cylinder or custom fitted insert can be made for individual patients. Such a fitted cylinder can be made to work in conjunction with a partial vacuum. For example, differing base units may be made for differing users with differing needs. In other embodiments, differing shaping elements and materials can be utilized. In yet other embodiments, electrotherapeutic devices can be configured to work in conjunction with the improved penile shaping system.

Having fully described at least one embodiment of the exemplary improved penile shaping device, other equivalent or alternative methods of implementing the improved penile shaping device according to the present invention will be apparent to those skilled in the art. Various aspects of the invention have been described above by way of illustration, and the specific embodiments disclosed are not intended to limit the invention to the particular forms disclosed. The particular implementation of the improved penile shaping device may vary depending upon the particular context or application.

By way of example, and not limitation, the improved penile shaping device described in the foregoing patent application is principally directed towards a penile shaping device. However, other techniques may instead be applied vacuum pump systems which implementations of the present invention are contemplated as within the scope of the present invention. For example, the improved penile shaping device can be utilized as a pump used for the treatment of other erectile dysfunction issues. Another use is for penile enlargement. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Although specific features of the invention are shown in some drawings and not others, persons skilled in the art will understand that this is for convenience. Each feature may be combined with any or all of the other features in accordance with the invention. The words “including,” “comprising,” “having,” and “with” as used herein are to be interpreted broadly and comprehensively, and are not limited to any physical interconnection. Claim elements and steps herein may have been numbered and/or lettered solely as an aid in readability and understanding. Any such numbering and lettering in itself is not intended to and should not be taken to indicate the ordering of elements and/or steps in the claims to be added at a later date.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Any amendment presented during the prosecution of the application for this patent is not a disclaimer of any claim element presented in the description or claims to be filed. Persons skilled in the art cannot reasonably be expected to draft a claim that would literally encompass each and every equivalent.

What is claimed is:

1. A penile shaping device comprising:
a. an attachable proximal end having an opening capable of interfacing with a
human body;
b. a cylindrical frame unit, the said cylindrical frame unit comprising one or more cylindrical section units, said cylindrical section units having angled ends; and
c. an attachable distal end.

2. The penile shaping device of claim 1 wherein when the attachable proximal end is attached to the proximal end of the cylindrical frame unit, the said cylindrical frame unit comprising one or more segments, and the attachable distal end is attached to the distal end of the cylindrical frame unit, the said cylindrical frame unit comprising one or more segments.

3. The penile shaping device of claim 1 wherein the penile shaping device includes one or more vacuum lines and forms a vacuum cylinder when assembled.

4. The penile shaping device of claim 1 wherein the cylindrical frame unit comprising one or more segments, said segments having angled ends creates one or more bends in the cylindrical frame unit when the one or more segments, said segments having angled ends are rotatable so as to create angles in the cylindrical frame unit.

5. The penile shaping device of claim 1 further comprising a sealing flange around the opening of the attachable proximal end capable of interfacing with a human body.

6. The penile shaping device of claim 1 wherein the attachable proximal end capable of interfacing with a human body is angled, the angle of the said proximal end may vary.

7. The penile shaping device of claim 1 wherein the distal end has an attachment point for one or more accessories to attach to the said distal end.

8. The penile shaping device of claim 7 wherein the one or more accessories is a weighted attachment.

9. The penile shaping device of claim 7 wherein the one or more accessories is a vacuum pump.

10. The penile shaping device of claim 7 wherein the one or more accessories is a handle for adjusting the penile shaping device.

11. A penile shaping system, said system comprising:
a. one or more attachable proximal ends, said attachable proximal ends having an opening for accommodating a human penis and capable of interfacing with a human body so as to create a sealing flange;
b. one or more cylindrical section units, the said cylindrical frame units comprising one or more segments, said segments having angled ends; and
c. one or more attachable distal ends; and
d. a vacuum line attachment.

12. The penile shaping device of claim 11 wherein the penile shaping device forms a vacuum cylinder when assembled.

13. The penile shaping device of claim 11 wherein when the attachable proximal end is attached to the proximal end of the cylindrical frame unit, the said cylindrical frame unit comprising one or more segments, and the attachable distal end is attached to the distal end of the cylindrical frame unit, the said cylindrical frame unit comprising one or more segments.

14. The penile shaping device of claim 11 wherein the cylindrical frame unit comprising one or more segments, said segments having angled ends creates one or more bends in the cylindrical frame unit when the one or more segments, said segments having angled ends are rotatable so as to create angles in the cylindrical frame unit.

15. The penile shaping device of claim 11 wherein the distal end has an attachment point for one or more accessories to attach to the said distal end.

16. The penile shaping device of claim 15 wherein the proximal end capable of interfacing with a human body is angled, the angle of the said proximal end may vary.

17. The penile shaping device of claim 15 wherein the one or more accessories is a weighted attachment.

18. The penile shaping device of claim 15 wherein the one or more accessories is a vacuum pump.

19. The penile shaping device of claim 15 wherein the one or more accessories is a handle for adjusting the penile shaping device.

20. A penile shaping method using a penile shaping device comprising an attachable proximal end capable of interfacing with a human body; a cylindrical frame unit, the said cylindrical frame unit comprising one or more cylindrical section units, said cylindrical section units having angled ends; and an attachable distal end; the method comprising the steps of:
a. a user creating a penile shaping device by selecting an attachable proximal end capable of interfacing with a human body for use, attaching the said attachable proximal end capable of interfacing with a human body to the one or more cylindrical section units, the said cylindrical frame units comprising one or more segments, said segments having angled ends, and selecting an attachable distal end and attaching the said attachable distal end to the one or more cylindrical section units, the said cylindrical frame units comprising one or more segments;
b. a user preparing the pubic area for attaching the penile shaping device;
c. a user placing a penis through the opening of the attached proximal end into the main cylinder;
d. a user creating a partial vacuum inside the penile shaping device, the partial vacuum attaching the penile shaping device to the pubic area;
e. a user manipulating the attached penile shaping device by manually grasping the said attached penile shaping device by
i. holding the penile shaping device at different angles for a recommended amount of time;
ii. applying one or more shaping forces to the attached penile shaping device by rotating one or more cylindrical section units in the attached penile shaping device;
iii. applying one or more distractive forces by allowing the attached distal end to freely hang or by manipulating the attached penile shaping device; and
f. a user releasing the said partial vacuum from inside the assembled angled proximal end and main cylinder.
